(19) United States
(12) Patent Application Publication
(10) Pub. No.: US 2017/0292123 A1
(43) Pub. Date: Oct. 12, 2017

(54) DEVICE FOR PURIFYING NUCLEIC ACIDS

(71) Applicant: AXAGARIUS GMBH & CO. KG, Duren (DE)

(72) Inventors: Hubert GREILER, Kettenis (BE); Markus MEUSEL, Wurselen (DE)

(21) Appl. No.: 15/634,449

(22) Filed: Jun. 27, 2017

Related U.S. Application Data
(62) Division of application No. 14/821,947, filed on Aug. 10, 2015.

(30) Foreign Application Priority Data
Aug. 13, 2014 (DE) 102014216016.2

(51) Int. Cl. C12N 15/10; B01D 15/38; B01D 15/22

(52) U.S. Cl. CPC C12N 15/1006; B01D 15/22; B01D 15/3819

(57) ABSTRACT

The invention relates to a device (1) for purifying nucleic acids composed of a one-piece hollow body (2) comprising an upper portion (3) having an inlet port (5) and a lower portion (4) having an outlet port (6), wherein within the hollow body (2) at the least one nucleic acid-binding matrix (7) is arranged, wherein the device (1) is characterized in that between the upper portion (3) and the lower portion (4) a predetermined breaking point (10) is provided and the nucleic acid-binding matrix (7) is arranged in the lower portion (4). The invention further relates to a method for producing such a device, a method for purifying nucleic acids by means of a device according to the invention, and a kit comprising a device according to the invention.

The invention relates to a device for purifying nucleic acids composed of an integrally formed hollow body comprising an upper portion having an inlet port and a lower portion having an outlet port, wherein at least one nucleic acid-binding matrix is arranged within the hollow body. The invention further relates to a method for producing such a device, a method for purifying nucleic acids using a device according to the invention, and a kit comprising a device according to the invention.

The preparation of nucleic acids is increasingly gaining importance. RNA and DNA are, for instance, needed in laboratories working in the fields of analytical medicine, biochemistry, and molecular biology. Applications range from gene technology, medicine, veterinary medicine, forensics, molecular biology to biochemistry, and from basic research to applied routine diagnostics.

Various methods are known for isolating nucleic acids from biological samples. In the historical methods the sample material is lysed (some by mechanical means or supported by enzymatic digestion) and the released nucleic acids are purified with phenol/chloroform mixtures. The purification can also make use of completely different technological methods, such as precipitation of the nucleic acids, or cesium chloride density gradient centrifugation.

All of the above-named methods have significant disadvantages, such as the use of harmful phenol/chloroform mixtures, or intensive post-purification of the isolated nucleic acids. For these reasons, new methods for isolating nucleic acids have been adopted within the past years. These include silica- and anion-exchange-based techniques. While to this day anion exchange is preferably used for DNA preparations of especially high purity (such as for transfection experiments), the silica-based technique has become popular as a simple and cost-effective method for a variety of applications.

It has been known since the 1950s that DNA reversibly binds to silicates and other inorganic carriers in the presence of chaotropic salts. The salts disrupt the hydrogen bonding network surrounding nucleic acids and create a hydrophobic microenvironment. Under these conditions the nucleic acids then bind to the silica matrix while proteins and other contaminants do not bind and are washed away. If, however, the binding solution is replaced by a low salt buffer or water, the nucleic acids become rehydrated and readily separate from the silica matrix from which they can be eluted. These methods are referred to as “bind-wash-elute” methods due to the sequence of nucleic acid-binding, one or more washing steps, and lastly the elution of the pure nucleic acids.

Spin columns containing a nucleic acid-binding silica membrane as solid phase or an alternate mineral binding matrix are commonly used for bind-wash-elution purification. Because the individual processing steps, for example, the binding of nucleic acid are commonly performed in small laboratory benchtop centrifuges, the corresponding columns are referred to as “mini spin” columns. Such columns are generally known in the state of the art and refer to columns that can, for example, be inserted into 1.5 mL Eppendorf reaction vials in which they can be processed in a microcentrifuge.

The manner the mini spin columns are constructed, however, poses several disadvantages. For example, the columns can generally accommodate only 1 mL of liquid so that larger amounts of liquids can be processed only in several subsequent steps. According to the state of the art, this disadvantage is circumvented by employing larger binding columns (usually referred to as L (large) columns or XL (extra-large) columns. The systems are commercially available by several manufacturers of nucleic acid purification kits, such as Sigma, MACHEREY-NAGEL, Qiagen or Promega and are well known to the person skilled in the art. The disadvantage of these columns, however, is that the larger diameter of the columns requires centrifugation to take place in large floor centrifuges. As opposed to the small benchtop centrifuges that are freely available for use in any laboratory, this poses a significant disadvantage in terms of handling, time expenditure as well as in the purification efficiency. For instance, the maximum number of columns that can be processed in parallel are limited in comparison to the small mini spin columns; another disadvantage is the large elution volume (which causes the nucleic acids to be “diluted”) and the large dead volume in the column (loss of nucleic acids).

Various technology approaches are currently known in the state of the art to circumvent these disadvantages.

DE 10 2004 034 474 A1 describes a device and method for nucleic acid purification in which a column body of a small spin column, such as a mini spin column, is enlarged by an attachable reservoir. The connection is generated by the geometrical design of the column intake and of the reservoir outlet. The connection is created by friction and pressure; it is not created by other means and is easily disconnectable. In this multi-component configuration, the reservoir remains attached to the spin column during the first processing steps, such as during loading of the column and the first wash steps. Since columns with an attached reservoir cannot be processed in a benchtop centrifuge, the first steps are performed using a vacuum chamber. To this end, the spin columns with their attached reservoirs are connected via the bottom outlet to a vacuum chamber. When a vacuum is applied to the chamber, the liquid is drawn from the reservoir, through the silica membrane, to the bottom of the column and into the vacuum chamber. The flow-through is generally discarded. The reservoir is removed only at the end of the procedure, and the column is then further processed in the centrifuge, for example, for the elution step.

A very similar approach is described in U.S. 2010/0222450; a reservoir to increase the volume is also present here as well as a small spin column, such as a mini spin column, that is connected to the reservoir via a multi-component design. However, unlike in DE 10 2004 034 474 A1 mentioned above, the reservoir is not simply placed onto the column, but rotatably connected by means of a bayonet lock. The upper inlet port of the centrifugation column is designed such that various types of reservoirs can be connected by means of a type of coupling system. The configuration of this coupling system is preferably designed as a luer/lock or luer/slip system. A syringe, for example, may also serve as reservoir; other embodiments comprise cylindrical hollow bodies having inlet-and outlet ports, the connection to the spin column being established via the outlet-side of the reservoir. In these embodiments, the coupling serves to connect the spin column and the reservoir.

Processing of small mini spin columns for the extraction of RNA from biological materials without the use of a volume-increasing reservoir is described in U.S. Pat. No. 6,218,531; there, the common mini spin columns are processed using vacuum following the “bind-wash-elute” procedure. Suitable vacuum chambers are known to the skilled person and commercially available (see, for example, Promega Vac-Man Laboratory Vacuum Manifold, Cat. No A7231, Promega Corporation, Madison, Wis., USA). Merely elution of the RNA bound to the silica membrane is performed in a benchtop centrifuge. The use of a reservoir or multi-piece component is not disclosed in U.S. Pat. No. 6,128,531. The disadvantage of this method is that the quantity of liquid that can be processed is limited by the restricted volume of the mini spin column.

EP 1049801 also describes the isolation of nucleic acids by means of plastic columns and vacuum. Various hydrophobic membranes are thereby arranged on a polyethylene frit that serves as a mechanical support within a plastic body. The column is connected via a luer connection to a vacuum chamber. All isolation steps take place under vacuum. The removal of the detached (eluted) nucleic acids occurs from the same side of the membrane from which they were introduced to the membrane.

DE 20 2004 006 675 U1 describes a device for purifying nucleic acids, with a first hollow body being reversibly connected to a second hollow body. The connection is formed, for example, as a screw connection, e.g., by an external or internal screw thread. In other embodiments, the two hollow bodies are connected together by pressure forces, for example, by a plug connection. The second hollow body is thereby a small spin column having a nucleic acid-binding material, while the first hollow body assumes the function of a reservoir, so that in this arrangement large amounts of liquid can be initially processed. A disadvantage of this arrangement is that due to the dimension of the reservoir the connected hollow body must be processed in a large floor centrifuge. The process is thus time- and labor intensive.

A very similar approach is described in EP 2055385. Here, a mini spin column containing the nucleic acid-binding material is connected via an adapter with a reservoir to form a multi-piece arrangement. Here, too, the processing is conducted in a large floor centrifuge. The mini spin column is separated from the reservoir only at the elution step and further processed in a benchtop centrifuge.

The use of a reservoir for increasing the volume without the need for reversibly and at least temporarily connecting multiple components is described in DE 29803712 U1. Here, too, a device for isolating nucleic acids is described. The one-piece construction is characterized by a large-volume reservoir in the upper part of the column and a small lower portion that contains the silica membrane. The one-piece construction eliminates the need for a gas- and liquid-tight connection between multiple components. The reservoir allows large-volume or highly diluted samples to be directly processed. The small silica membrane in the lower part of the column allows for small elution volumes and reduces the dead volume. A disadvantage here, however, is that due to the large configuration of the reservoir component the column can be processed only in large floor centrifuges.

As can be seen in the art, a variety of devices and methods with multi-part embodiments exist. To overcome the disadvantage of large-sized columns, small mini spin columns are connected with reservoirs in various configurations to increase the volume. In these multi-part embodiments, various connection techniques are employed in order to connect the mini spin columns with the reservoir for nucleic acid-binding.

One-piece embodiments have either the disadvantage of their small volume that makes a large number of loading steps necessary or that they accept the disadvantage of a large column diameter that excludes processing in simple benchtop centrifuges.

It is therefore an object of the present invention to provide a device for purifying nucleic acids of the above-mentioned type that allows the processing of larger quantities of liquid while at the same time being easy to use, and in particular allows further processing in a benchtop centrifuge.

The object is achieved by a device for purifying nucleic acids that is composed of a one-piece hollow body comprising an upper portion having an inlet port and a lower portion having an outlet port, wherein within the hollow body at least one nucleic acid-binding matrix is arranged, and wherein the device is characterized in that between the upper portion and the lower portion a predetermined breaking point is provided and the nucleic acid-binding matrix is arranged in the lower portion.

In other words, a reservoir filter column in a one-piece configuration is provided having a predetermined breaking point in which the portion above the predetermined breaking point (“reservoir”) serves to accommodate larger volumes of sample that, after processing of large sample volumes and optional further washing steps, can be removed, for example, by manual breakage. Predetermined breaking points are commonly employed in medical pharmaceutical packaging and containers. For example, EP 1136380 describes a disposable container for medical or pharmaceutical uses having a circumferential predetermined breaking point. Such predetermined breaking points can also be employed in the context of the present invention.

The portion above the predetermined breaking point, which is also hereinafter referred to as “reservoir,” may simultaneously act as a lever during the separation of the reservoir and the lower portion at the predetermined breaking point, so that an additional tool for separation is not required. After breaking off the upper portion, i.e., the reservoir, the lower portion comprising the nucleic acid-binding matrix loaded with nucleic acids can be further processed in a table top centrifuge. The reservoir can be discarded after it is removed from the lower portion.

Unlike two-component arrangements that must be joined or screwed together before use, this additional step is omitted in the device according to the invention, thereby simplifying handling.

In addition, with the device according to the invention it is ensured that the connection between the reservoir and the lower portion is gas-tight and liquid-tight. The known multi-part designs must ensure that the individual components and parts are connected in a manner that renders them gas- and liquid-tight. This is essential for correct operation under vacuum. The negative pressure at the column outlet draws in the liquid from the reservoir and through the nucleic acid-binding matrix when the atmospheric pressure acts as the driving force on the liquid via the open side of the reservoir. If, due to leaks or leaking connections, the atmospheric pressure acts below the liquid surface, the flow and thus the filtration/binding is reduced or interrupted. In addition, unwanted contaminations may also result. This problem may occur with the well-known two-piece configurations, for example, when the connection between the two components is not completely sealed, either because of insufficient precision in manufacturing of the components or by faulty assembly.

In the context of the present invention, a “nucleic acid-binding matrix” is to be understood as a solid material that is suitable to separate nucleic acids from a liquid matrix. The separation can be carried out mechanically, such as with a filter, by means of physical and/or chemical interactions such as adsorption. Combined mechanisms of nucleic acid-binding, such as mechanically and by adsorption, are therefore explicitly included.

It is understood that the statement of purpose “for the purification of nucleic acids” is not meant to be limiting but that in the present invention all devices are protected that are in principle suitable for this purpose, even if they are ultimately used for other purposes. Thus, the device may be used, for example, for the separation of biomolecules of any kind, or as a filter, or for the separation of non-biomolecules.

The device according to the invention comprises the hollow body in which the nucleic acid-binding matrix is arranged, whereby said device may comprise in addition further components, such as, for example, seals, frit, devices for fixing the nucleic acid-binding matrix and the like.

Another object of the present invention relates to a method for producing a device according to the invention for purifying nucleic acids, with which a one-piece hollow body having an inlet opening and an outlet opening and a predetermined breaking point is produced and at least one nucleic acid-binding matrix is subsequently arranged within the hollow body between the predetermined breaking point and the outlet opening.

The present invention further relates to a method for purifying nucleic acids from a liquid nucleic acid-containing sample comprising the steps of:
a) Providing the sample containing the liquid nucleic acids and adjusting the binding conditions so as to achieve binding of the nucleic acids to the nucleic acid-binding matrix;
b) Transferring the sample into a device according to the invention through the inlet opening of the device;
c) Passing the sample through the nucleic acid-binding matrix, whereby the nucleic acids bind to the nucleic acid-binding matrix and whereby the passage is effected in particular by applying a vacuum to the outlet opening of the device;
d) Optionally washing the nucleic acid-binding matrix;
e) Separating the upper portion from the lower portion along the predetermined breaking point, in particular by manual breakage;
f) Optionally washing the nucleic acid-binding matrix;
g) Eluting the nucleic acids from the nucleic acid-binding matrix and collecting the eluted nucleic acids in a separate collection vial, with the elution being preferably carried out in a centrifuge.

The invention also relates to a kit for purification of nucleic acids from a nucleic acid-containing liquid sample comprising a device according to the invention, as well as to an instruction manual for performing the method according to the invention, and/or to that are means suitable to purify nucleic acids, such as at least one lysis- and/or binding buffers, wash buffer and/or elution buffers.

According to a preferred development of the device according to the invention, the volume of the upper portion corresponds to a volume having at least a 5-fold larger volume of the lower portion, particularly at least 20-fold, preferably at least 40-fold, more preferably at least 50-fold. Embodiments having a 55-fold volume and greater are also possible. In the context of the present invention volume is to be understood as the void volume in the respective portion of the device. In a cylindrically shaped upper portion its volume is, for example, the volume enclosed by the cylinder delimited by the rim of the inlet opening and delimited on the opposite side by the predetermined breaking point.

The different volumes of the upper and lower section can be realized for example, by choice of different diameters and/or different linear expansion of the upper and lower section.

In absolute terms, the volume of the lower portion can substantially correspond to the volume of commercially available mini spin columns, thus, for example, about 1 mL, especially 0.5 to 1.5 mL. The volume of the upper portion, i.e., of the reservoir, may independently thereof be, for example, 5 to 100 mL, especially 10 to 80 mL, preferably 20 to 40 mL, although other volumes are possible, if required for certain applications. The size of the reservoir in particular can vary within a wide range without requiring significant adjustment of the further processing steps when using the device according to the invention, since the reservoir is separated at the predetermined breaking point prior to centrifugation.

In the device according to the invention, the upper portion and the lower portion may each independently exhibit a round or rectangular, e.g., a square, cross-section. To simplify production, the choice of a round cross-sectional shape is preferred.

The upper portion and the lower portion may independently of one another exhibit, for example, a cylindrical or conical shape. In particular, the upper portion may exhibit a widening conical shape expanding in the direction of the inlet opening, thereby facilitating the filling of larger sample volumes.

According to another preferred embodiment of the device according to the invention, the upper portion has a substantially cylindrical configuration, with the cross-section above the predetermined breaking point tapering off in the direction of the lower portion, preferably such that the outer diameter of the upper portion tapers off to about the outer diameter of the lower portion immediately below the predetermined breaking point, preferably in the form of conical tapering. This is advantageous because in this way it can be ensured that the liquid sample filled in the reservoir can flow virtually residue-free into the lower portion.

It is further advantageous that immediately below the predetermined breaking point the lower portion is provided with at least one tapering having a step, in particular with a tapering having two steps. The step-wise tapering forms locking faces on the outside of the lower portion by means of which, after the upper portion is removed, the lower portion can be inserted into the holes of a centrifuge, in particular of a benchtop centrifuge. The inner wall of the lower portion in the area of the outer side of the step-wise tapering is thereby preferably not configured in a step-wise fashion, but provided with an inclined tapering. This prevents, as far as possible, residues of the fluid sample from adhering.

According to an advantageous development of the device according to the invention, the upper portion around the inlet opening is provided with a circumferential rim lip that provides the device with higher stability. In addition, the rim lip may serve as a stop surface that allows inserting the device into a mounting hole having a hole diameter which substantially corresponds to that of the upper portion but is smaller than the outer diameter of the rim lip.

The hollow body may in principle be constructed from any suitable material. Suitable materials should have a certain mechanical stability, be as inert as possible to the chemicals typically used, and exhibit low nucleic acid-binding. Their mechanical properties should also permit easy separation of the upper and lower part at the predetermined breaking point. Preferably, the hollow body is constructed of plastic. Suitable plastics may be selected from thermoplastics, duroplastics and elastomers. The plastic is in particular selected from polyolefins such as polyethylene or polypropylene, from bio-based plastics such as polyhydroxybutyric acid or polylactates, polyamides such as nylon, polyimides, acetals, polyvinyl chloride, polytetrafluoroethylene, polyesters, polycarbonates, polymethyl(meth)acrylates, acrylonitrile-butadiene-styrene terpolymer (ABS), polystyrene or any desired mixtures and/or copolymers thereof.

Furthermore, the hollow body may be provided, at least partially or completely, in particular on its inner surface, with a coating, which reduces or increases, for example, the surface tension with respect to water. Such coatings can be conducted in a conventional manner, for example, by silanization of the surface or other measures known to the expert.

The hollow body can, for example, be manufactured via an injection molding process, blow molding, casting technique with silicone forms, or methods of rapid prototyping (e.g., 3D printing, fused deposition modeling, laser sintering, or electron beam melting). An injection molding process is advantageous in that it allows the hollow body to be manufactured with sufficient precision, high reproduction accuracy, and in large quantities. In addition, it is comparatively simple to integrate the predetermined breaking point in the injection molding process. The predetermined breaking point may naturally also be introduced after the hollow body has been manufactured, for example, by lathe cut action, or by means of a laser.

Alternatively, the hollow body of the invention can also be produced by forming machining methods; these include, for example, turning, milling, drilling, sawing, and grinding.

Any material that is known in the art for the stated purposes can be employed as nucleic acid-binding matrix in the context of the present invention. Examples of mineral materials suitable for this purpose are porous or non-porous substances such as silica, glass, quartz, zeolites or metal oxides. The materials may be provided in the form of membranes, fibers, fabrics, sinters, sieves, or particulates. Equally suitable are anion exchange resins or materials chemically modified to comprise anion exchange functionalities. These may be used as a porous membrane, in particulate form, or in other manners known per se. Of the mineral carriers, in particular glass fiber-quartz fiber membranes are attractive because they are inexpensive to manufacture and exhibit good nucleic acid-binding properties. Glass fiber filters/membranes typically consist of micro-glass fibers. In the technical field, such filters are usually employed as water or air filters. The binding matrix may also comprise particulate material (silica particles, silica gel). Particulate material is preferably fixed between two liquid-permeable layers (e.g., plastic frit or filter) that prevent particles from passing through.

If membranes or filters are used, the pore size of the membrane can be adapted to the particular application. Due to the manner a fiber network is constructed, it is generally not possible to define a pore size for glass fiber membranes or -sheets, even though the literature usually states values, for example, of from 2-5 μm. The filter efficiency of such materials is therefore usually defined by its retention ability in accordance with DIN EN 1822-3 (January 2011). Customary retention values (i.e., particle sizes that are retained) are, for example, in the range of from 0.1 to 5 μm, preferably of from 0.5 to 4 μm, more preferably of from 1 to 3 μm.

According to a preferred embodiment of the device according to the invention, the nucleic acid-binding matrix may be immobilized on a support. Particularly suitable for this purpose is a support frit comprised of, for example, glass, ceramic or plastic whereby in principle the same materials can be used as the materials from which the hollow body is constructed; however, the materials can be chosen irrespective of those the hollow body is manufactured of. A preferred embodiment provides for the support frit to be manufactured of sintered plastic. Fixing of the nucleic acid-binding matrix on the support can, for example, be accomplished via a clamping ring that is fixed inside the hollow body by clamping, gluing, or in other ways. This assembly makes it possible to produce and/or offer different nucleic acid-binding matrices for the same hollow bodies. The assembly can then be performed as needed, and even by the actual consumer.

According to a particularly preferred embodiment of the device according to the invention, the predetermined breaking point is formed by a weakening line that substantially encircles the hollow body in a continuous form, in particular as a groove reducing the wall thickness of the hollow body. It is particularly preferred for the groove to have the shape of a keyway since this allows particularly easy separation, i.e., without great effort, of the upper and lower portions from each other.

It is very particularly preferred for the predetermined breaking point to be designed such the upper portion is separable from the lower portion without the aid of tools.

In the context of the inventive method in particular those steps that require a large volume of liquid can be carried out under vacuum in commercially available vacuum chambers, for example, by applying a vacuum of 500 mbar or less, in particular of 300 mbar or less. The outlet opening of the device may thereby be configured so as to fit standard vacuum chambers with standard adapters. Preferably, luer/lock or luer/slip adapters known to those skilled in the art are employed. To this end, in a preferred embodiment of the device according to the invention the outlet opening is designed such that it is suitable for attachment to a vacuum chamber, with the outlet opening being preferably configured as a luer male adapter. This is particularly advantageous because in this form the necessity of using adapters or the like is circumvented and the lower portion can be attached directly onto the vacuum chamber connector.

Any biological material containing nucleic acids is suitable as sample material, such as animal or human tissue, pieces of tissue, body fluids such as saliva, sputum, cerebrospinal fluid, whole blood, serum, or plasma. Likewise, bacteria, yeasts and other fungi, or viruses are to be understood as “sample material” as well as PCR amplification reactions containing primers and DNA fragments, or cell culture supernatants. Sample material may also include environmental or food samples. Artificial sample material, e.g., material containing synthetic or in-vitro generated nucleic adds, falls within the scope of the present invention.

Nucleic acids within the meaning of the invention include any form of nucleic acids: RNA and DNA of bacterial, viral, animal or plant origin. Furthermore, nucleic acids are to be understood as long- and short-chained, single- and double stranded, linear and branched natural nucleic acids (e.g., genomic DNA, mRNA, miRNA, siRNA), as well as artificial nucleic acids, such as PCR fragments, as well as free or cell-bound nucleic acids or those generated in vitro (e.g., cDNA).

The method according to the invention is not limited to a particular technique of nucleic acid purification. Various methods can be found in the prior art that can be employed and are known to the skilled person. These include, for example, the use of anion exchangers, the use of chaotropic salts, the use of anti-chaotropic salts, precipitations (e.g., precipitation with polyethylene glycol), filtration, exploiting hydrophobic interactions for nucleic acid-binding and other processes.

A particularly preferred embodiment of the device and/or the method according to the invention involves the use of chaotropic salts in combination with a silica membrane as nucleic acid-binding matrix. In accordance with current theories, the chaotropic salts disrupt the ordered water structure surrounding the nucleic acids, allowing them to bind to surfaces of mineral carriers, especially to glass and silica carriers (silicon dioxide in the form of fibers or particles, glass fiber, silica gel, zeolite, etc.). Chaotropic salts are defined as having the ability to denature proteins, increase the solubility of non-polar substances in water, and destroy hydrophobic interactions. The strength of the chaotropic character of a salt is described by the so-called Hofmeister series. In the context of the present invention, for example, sodium perchlorate, sodium iodide, guanidine isothiocyanate and guanidine hydrochloride or combination thereof may be employed as chaotropic salts.

In the context of the method of the invention the biological sample can first broken open, i.e., lysed so as to release the nucleic acids from the material. Lysing may be accomplished by a mechanical, chemical and/or enzymatic digestion. Lysing of the sample is often supported by an appropriate buffer chemistry that includes, for example, detergents. Suitable lysis conditions are known to the person skilled in the art.

If the opened (lysed) material with its released nucleic acids is added to the inventive device, the sample passes through the nucleic acid-binding matrix. This can be accomplished, for example, by gravity, centrifugation, vacuum, and pressure. For this process step, the device is preferably attached to a vacuum chamber, which, upon application of an appropriate negative pressure, draws the liquid out of the reservoir and through the nucleic acid-binding matrix into the vacuum chamber. During this step, the nucleic acids bind to the nucleic acid-binding matrix. The binding in the presence of the above-described chaotropic salts (common salt concentrations are e.g., of from 1 to 6 mol/L), optionally supported by other components of the binding solution (such as short-chain alcohols, including methanol, ethanol, propanol and the like), is known to the skilled person. Other binding principles, such as the binding to the anion exchanger, are also possible.

After this step, the nucleic acids bound to the nucleic acid-binding matrix are present in the lower portion of the device according to the invention, while the sample liquid was removed. The lower portion containing the nucleic acids may now be separated from the upper portion of the device for further processing. The separation is performed by simple breakage at the designated predetermined breaking point. The lower portion of the column containing the bound nucleic acids is further processed, while the reservoir is discarded. Conveniently, the separation is carried out after the washing steps or prior to elution.

Since impurities that also bind to the solid phase during the binding step typically remain bound to the nucleic acids, a washing step may conveniently follow this step. Such wash steps, and suitable solutions are known to the skilled person. Washing conditions are typically adjusted so as to avoid disrupting the nucleic acid-binding to the solid phase, i.e., to the nucleic acid-binding matrix while impurities are removed. In the example concerning the use of chaotropic salts, the binding step is usually followed by a washing step with high (chaotropic) salt concentration in which detergents and proteins are removed, as well as a further washing step with no/low salt and high alcohol concentration. This step removes remaining chaotropic salts while the binding of nucleic acids to the solid phase is maintained in the presence of high concentrations of alcohol. A high (chaotropic) salt concentration in this context is understood as being, for example, an aqueous solution of at least 0.2 mol/L salt, preferably of at least from 0.5 mol/L to 5 mol/L. The second “low salt” washing step is understood as meaning a salt concentration of at most 1 mol/L, preferably at most 0.1 mol/L, and a “high concentration of alcohol” is understood as being an alcohol concentration of at least 50 wt.-%, preferably from 60 wt.-% to 90 wt.-%.

Depending on the degree of contamination, these washing steps can be performed prior to separation of the lower part of the device from the reservoir. It is thereby advantageous that large volumes of washing solutions are applied in a single step and can be passed through the nucleic acid-binding matrix. Before eluting the nucleic acids, at the latest, the reservoir of the inventive device can be separated from the lower portion containing the bound nucleic acids.

After separation of the reservoir a further wash step is preferably performed in a centrifuge. Due to the absence of the reservoir, the lower portion of the device representing the actual column can be processed in conventional benchtop centrifuge. The high accelerations achievable in a conventional benchtop centrifuge can easily and completely remove remnants of the washing solutions (e.g., alcohols) as would not or not as completely and effectively be accomplished under vacuum.

In a further preferred embodiment of the method according to the invention it is provided to not load washing solution onto the column prior to elution but to briefly centrifuge the column (for example, for 2 minutes at 11,000×g) so as to remove remnants of the washing solutions. If for the binding of nucleic acids known chaotropic chemistry is employed, the elution is usually performed with buffers of low ionic strength (e.g., 5 mM Tris buffer) or water. The water rehydrates the nucleic acids so that are able to detach from the solid phase and can be collected in a separate receptacle during centrifugation.

If another method is used to bind the nucleic acids, such as anion exchange, the washing and elution of the nucleic acids is performed using different conditions. These methods are known in the art to the skilled person. Anion exchangers are predominantly employed for plasmid purification on the so-called midi or maxi scale. Bacterial cells are harvested by centrifugation, taken up in resuspension buffer, and lysed under alkaline conditions. Under these conditions, both chromosomal and plasmid DNA denatures. The addition of potassium acetate leads to neutralization and to the formation of a precipitate of chromosomal DNA, cellular debris, and protein. Only the plasmid DNA is able to renature and remains in solution. After separation of the insoluble components, the bacterial lysate is applied to the anion exchanger. At a high salt concentration and low pH the negatively charged DNA backbone binds to the positively charged anion exchange groups. Washing steps with increasing salt concentration remove contaminants. Lastly, the bound DNA is eluted from the anion exchanger by increasing the pH. The DNA is subsequently purified from the salt residues by precipitation.

The present invention will be discussed in more detail below with reference to two drawings and embodiments. It is shown in

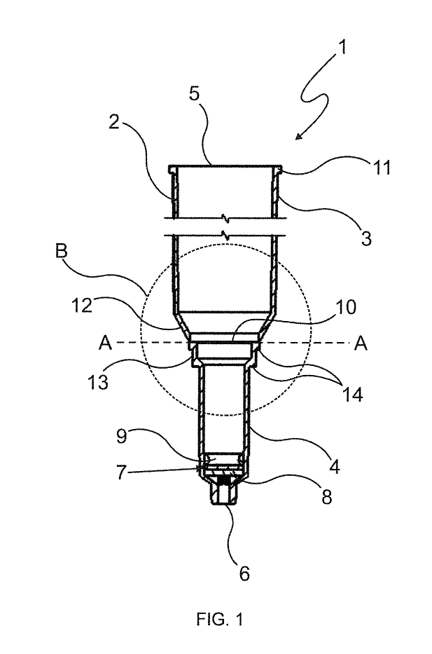

FIG. 1 an embodiment of an inventive device,

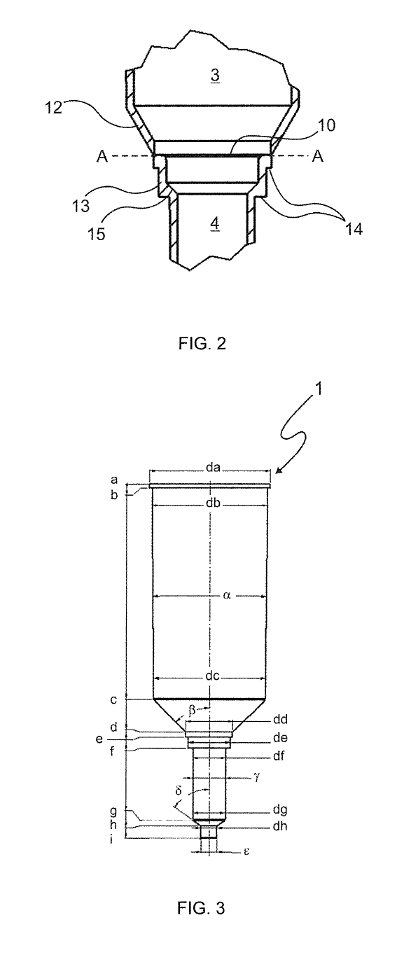

FIG. 2 a sectional enlargement of a portion B of the device of FIG. 1, and

FIG. 3 a precise embodiment of the device of FIG. 1 with dimensional data.

In FIG. 1 a device 1 for isolation of nucleic acids is shown in a lateral sectional view. The device comprises an integrally formed hollow body 2 with a round cross section and an upper portion 3, as well as a lower portion 4. Hollow body 2 is constructed of polypropylene as a molded part. At upper portion 3 an inlet opening 5 is provided for introducing a liquid sample containing nucleic acids. In lower portion 4, a nucleic acid-binding matrix in the form of a silica membrane 7 (glass fiber filter) with a retention of 1.4 μm according to DIN EN 1822-3 (January 2011) is fixed on a carrier frit 8 by means of a clamping ring 9. In flow direction of the device 1, an outlet opening 6 is provided below silica membrane 7 that is configured as a luer male adapter.

Between upper portion 3 and lower portion 4 a predetermined breaking point 10 is formed in form of a circumferential key groove, which allows manual separation of upper portion 3 and lower portion 4 along the dashed line A-A. Upper portion 3 has a void volume of 38.5 mL, lower portion 4 has a void volume of approximately 1 mL of which typically 0.7 mL is usable so as to prevent liquids from spilling over the rim.

Upper portion 3, which is also referred to as a reservoir, comprises a circumferential lip 11 at inlet opening 5. At its opposite end, i.e., at the side facing predetermined breaking point 10, top portion 3 is provided with a conical tapering 12. By means of conical tapering 12, the outer diameter of upper portion 3 is reduced up to predetermined breaking point 10 to about the outer diameter of the lower section 4.

As is shown in FIG. 2, an enlarged circular detail B of FIG. 1, lower portion 4 is provided immediately below predetermined breaking point 10 with a two-step tapering 13 which forms latching surfaces 14 on the outer side of lower portion 4 that, after removal of upper portion 3, allows lower portion 4 to be inserted in a centrifuge. The inner wall of lower portion 4 in the area of the outer side of the stepwise tapering 13 is thereby preferably not provided stepwise, but with an inclined tapering 15.

FIG. 3 shows a specific embodiment of device of 1 according to FIG. 1 with dimensional data. The respective dimensions are summarized in the following table:

| | Length to a position x | | dx | | |
|---|---|---|---|---|---|
| Position | [mm] | Diameter | [mm] | Angle | [°] |
| a | — | da | 32 | α | 1° |
| b | 1 | db | 30.6 | β | 45° |
| c | 56.4 | dc | 29.6 | γ | 1.1° |
| d | 65 | dd | 12.4 | δ | 55° |
| e | 66.4 | de | 11.1 | ε | 1.7° |
| f | 69.3 | df | 8.8 | | |
| g | 88.4 | dg | 8.4 | | |
| h | 89.8 | dh | 4.3 | | |
| i | 93.2 | | | | |

The length values in the table indicate the distance of the respective position to the top edge of device 1, the position a. The length of the sections can be calculated from the distance between the respective end section and the end of the previous section. For example, the section a-b extends from the beginning of rim lip 11—position a—to the end of rim lip 11—position b. The diameters values are based on the position shown in FIG. 3. The angles refer to the angle between the wall at the designated position and the imaginary geometric center line extending continuously through the device 1 in flow direction. The hollow volumes of upper and lower portions 3, 4 correspond to those shown in FIG. 1.

In the following, several examples of embodiments are described for separating nucleic acids using inventive device 1 and with the method according to the invention.

### EXAMPLE 1

### Protocol for Purification of Plasmid DNA from E. Coli

1. 200 mL of an E. coli XL1 Blue culture with low-copy vector and an OD of 1.8 was pelleted and the cells resuspended in 7 mL A1 with RNase A. (A1: NucleoSpin plasmid, REF 740855, MACHEREY-NAGEL, Düren, Germany—commercial Tris/EDTA resuspension buffer containing RNase A).

2. After alkaline lysis with 7 mL A2 and neutralization with 8.4 mL of A3, the precipitate was removed by centrifugation (10 min, 10,000×g; NucleoSpin plasmid, REF 740588, MACHEREY-NAGEL, Düren, Germany—A2 commercial buffer for alkaline lysis of bacteria containing NaOH/SDS; A3 commercial neutralization- and binding buffer containing potassium acetate and guanidine hydrochloride).

3. The clear lysate was loaded by vacuum onto the column according to the invention and washed with 5 mL Wash Buffer AW, and 2×5 mL Wash Buffer A4 under vacuum (NucleoSpin plasmid, REF 740855, MACHEREY-NAGEL, Düren, Germany—AW commercial high salt wash buffer containing guanidine hydrochloride and ethanol; A4 commercial alcohol wash buffer containing 80% ethanol). The column according to the invention was designed in accordance with FIG. 1 with a predetermined breaking point in circumferential direction. Six layers of silica membrane were placed in the lower part of the column and immobilized in the column by means of a plastic clamping ring.

4. Then, the reservoir part was removed (broken off), and the lower part of the column was transferred to a collection tube (2 mL).

5. The column was dried in a benchtop centrifuge for 2 min at 11,000×g.

6. The DNA was eluted in 50 μL of elution buffer AE (5 mM Tris/HCl) by centrifugation for 1 min at 11,000×g.

### Result

The photometric measurement of the eluate determined a yield of 11.8 μg and purity of A260/A280=1.84 or A260/A230=2.23.

Without the column according to the invention the lysate would have needed to be loaded onto a commercially available mini spin column in approx. 30 individual steps of 700 μL each.

### EXAMPLE 2

### Protocol for Purification of PCR Fragments

1. A band weighing 5 g containing 20 μg of plasmid DNA was excised from a 1% TAE agarose gel (30 minutes, 90V) and incubated in 10 mL NTl buffer (NucleoSpin Gel and PCR Clean-up, REF 740609, MACHEREY-NAGEL, Düren, Germany—commercial binding buffer containing guanidine thiocyanate) at 55° C. until it was completely dissolved.

2. The sample was loaded under vacuum onto the column according to the invention (2 layers of silica membrane, polyethylene frit as a carrier) and washed with 5 mL Wash Buffer NT3 under vacuum (NT3, NucleoSpin Gel and PCR Clean-up, REF 740609, MACHEREY-NAGEL, Düren, Germany—commercial washing buffer containing 80% ethanol).

3. Then, the reservoir part was removed (broken off), and the lower part of the column transferred to a collection tube (2 mL).

4. The column was dried in a benchtop centrifuge for 2 min at 11,000×g.

5. The DNA was eluted in 100 μL of elution buffer NE (5 mM Tris/HCl) by centrifugation for 1 min at 11,000×g.

### Result

The photometric measurement of the eluate determined a yield of 12.8 μg (64%) and purity of A260/A280=1.85 or A260/A230=2.06.

Without the column according to the invention, the lysate would have needed to be loaded onto a commercially available mini spin column in approx. 20 individual steps of 700 μL each.

### EXAMPLE 3

### Protocol for Purification of Genomic DNA from Complex Biological Samples

1. 1 g of cooked ham was incubated in 2750 μL lysis buffer CF and 50 μL proteinase K for 3 h at 65° C. (lysis buffer CF: NucleoSpin Food, REF 740945, MACHEREY-NAGEL, Düren, Germany—commercial SDS-lysis buffer).

2. Undigested sample material was pelleted at 10,000×g for 10 min. To the clear supernatant 1 volume of binding buffer C4 and one volume of ethanol was added (C4: NucleoSpin Food, REF 7409454, MACHEREY-NAGEL, Düren, Germany—commercial binding buffer containing guanidine hydrochloride).

3. The sample was loaded under vacuum onto the column according to the invention (3 layers of silica membrane, polyethylene frit as carrier) and washed with 5 mL Wash Buffer CQW, 2×5 mL Wash Buffer C5 under vacuum (NucleoSpin Food, REF 740945, MACHEREY-NAGEL, Düren, Germany—CQW: commercial wash buffer containing guanidine hydrochloride and ethanol; C5 commercial alcohol wash buffer containing 80% ethanol).

4. Then, the reservoir part was removed (broken off), and the lower part of the column transferred to a collection tube (2 mL).

5. The column was dried in a benchtop centrifuge for 2 min at 11,000×g.

6. The DNA was eluted in 2×100 μL of elution buffer CE (5 mM Tris/HCl) by centrifugation for 1 min at 11,000×g.

### Result

The photometric measurement of the eluate determined a yield of 120 μg and purity of A260/A280=1.91 or A260/A230=2.19.

Without the column according to the invention, the lysate would have needed to be loaded onto a commercially available mini spin column in approx. 15 individual steps of 700 μL each.

### EXAMPLE 4

### Protocol for Purification of Genomic DNA from Human Plasma

1. 2.4 ml of plasma was mixed with 3.6 ml of buffer BB (binding buffer BB: NucleoSpin Plasma, REF 740900, MACHEREY-NAGEL, Düren, Germany—commercial binding buffer containing guanidine thiocynate and ethanol).

2. The sample was loaded under vacuum onto the column according to the invention (3 layers of silica membrane, polyethylene frit as a carrier). The following washing steps were carried out in a benchtop centrifuge.

3. The reservoir of the column was removed (broken off), the lower part of the column inserted into a 2 mL collection tube, 500 μL wash buffer WB added (commercial wash buffer, Tris/HCl, >60% ethanol) and centrifuged for 30 seconds at 11.000×g.

4. The collection tube containing the flow-through was discarded and washing performed a second time with 250 μL wash buffer WB.

5. The column was then dried by centrifugation at 11,000×g for 3 min.

6. The DNA was eluted in 200 μL elution buffer BE (5 mM Tris/HCl) by centrifugation for 30 sec. at 11,000×g.

In parallel, 2 further samples were processed. To one sample was added 240 μL plasma and 360 μL binding buffer BB following the NucleoSpin Plasma XS High Sens protocol (MN, REF 740900). This corresponds to 1/10 of the volume of the example in the above-mentioned embodiment. As a binding column, the conventional NucleoSpin Plasma XS mini spin column was used. The loading and all other steps were carried out in a tabletop centrifuge under identical conditions.

Another sample was prepared as described in the example of the above embodiment by adding 3.6 mL binding buffer BB to 2.4 mL plasma. Here, however the column used was a large funnel column (MACHEREY-NAGEL Funnel Column). The samples were processed using a standing floor centrifuge. The individual steps were as follows:

1. Wash step 5 mL WB (1,000×g, 3 min)

2. Wash step 2.5 mL WB and drying (3,000×g, 3 min)

3. Elution: 200 μL BE, 3,000×g 3 min.

The DNA was quantified by quantitative rtPCR (CyNamo Capillary SYBR Green qPCR Kit).

### Result

DNA was isolated from plasma using all three formats. The DNA yield using the NucleoSpin Plasma XS was 13 ng, 108 ng using the large funnel column (Funnel Column), and 194 ng using the column according to the invention. Compared to the mini spin column the 10-fold amount of plasma could be processed with the column according to the invention, (240 μL vs. 2.4 mL, 13 ng vs. 194 ng DNA yield). The inventive method is also superior to the large funnel column using the same sample volume (in both cases, 2.4 mL), in terms of DNA yield (194 vs. 108 ng), and DNA concentration (0.97 vs. 0.54 ng/μL).

### EXAMPLE 5

### Protocol for purification of RNA

1. RNA was purified by a clean-up protocol. The RNA was present in pre-cleaned form in water at a concentration of 1 ng/μL. 3 ml of the RNA-solution was mixed with 3.0 ml of buffer RCU (binding buffer RCU: NucleoSpin RNA Clean-up XS Kit, REF 740903, MACHEREY-NAGEL, Düren, Germany—commercial binding buffer containing guanidine thiocynate and ethanol).

2. The sample was vacuum-loaded onto the column according to the invention (3 layers of silica membrane, polyethylene frit as a carrier). The following washing steps were carried out in a benchtop centrifuge.

3. The reservoir of the column was removed (broken off), the lower part of the column inserted into a 2 mL collection tube, 400 μL wash buffer RA3 added (commercial wash buffer, Tris/HCl,>70% ethanol) and centrifuged for 30 seconds at 11,000×g.

4. The collection tube containing the flow-through was discarded and washing performed a second time with 200 μL wash buffer RA3.

5. The RNA was eluted in 100 μL water by centrifugation for 30 sec. at 11,000×g,

In parallel, 300 μL of the RNA solution was mixed together with 300 μL RCU and processed with conventional mini spin columns from the NucleoSpin RNA kit ((MACHEREY-NAGEL, Düren, Germany, REF 740955) by centrifugation. All remaining steps were carried out according to the protocol described above.

RNA was quantified using RiboGreen.

### Result

The RNA yield using the columns according to the invention was 5.5 μg while the yield using the mini spin columns was 0.7 μg. To some extent, the scale-up by the factor of 10 with respect to the starting material (300 μL with the mini spin column, 3 mL with the inventive column) is thus also reflected in the RNA yield.

### EXAMPLE 6

### Protocol for the Precipitation and Purification of Plasmid DNA

1. To 6 μg pcDNA3.1 in 5 mM Tris/HCl water was added to obtain 1, 2, 4, 8, 16 mL.

2. The DNA of the samples is precipitated by addition of 1 volume of polyethylene glycol (20% PEG 8000, 2.5 M NaCl) and incubation for 2 h at 4° C.

3. The reaction mixture was applied to the column according to the invention and drawn through by applying a vacuum (−300 mbar).

4. The reservoir of the column was removed (broken off) and the column was washed under vacuum by addition of 2×700 μL wash buffer A4 (commercially available alcohol wash buffer containing 80% ethanol).

5. The lower part of the column was inserted into a 2 mL collection tube and dried by centrifugation for 30 s at 11,000×g in a benchtop centrifuge.

6. The DNA was eluted in 2×200 μL of 5 mM Tris/HCL by centrifugation for 30 sec. at 11,000×g.

### Result

The DNA yield for 1, 2, 4, 8, and 16 mL was 5.8, 5.4, 4.8 and 4.5 μg DNA. Thus, yields between 99-72% were obtained.

1.-15. (canceled)

16. A method for purifying nucleic acids from a liquid nucleic acid-containing sample comprising
a) providing a device composed of a one-piece hollow body comprising an upper portion having an inlet port and a lower portion having an outlet port, wherein at least one nucleic acid-binding matrix is arranged within the hollow body, wherein between the upper portion and the lower portion a predetermined breaking point is provided and the nucleic acid-binding matrix is arranged in the lower portion;
b) providing the liquid nucleic acid-containing sample and adjusting the binding conditions so as to achieve binding of the nucleic acids to the nucleic acid-binding matrix;
c) transferring the sample into the device through the inlet opening of the device;
d) passing the sample through the nucleic acid-binding matrix, wherein the nucleic acids bind to the nucleic acid-binding matrix;
e) optionally washing the nucleic acid-binding matrix;
f) disconnecting the upper portion from the lower portion along the predetermined breaking point;
g) optionally washing the nucleic acid-binding matrix;
h) eluting the nucleic acids from the nucleic acid-binding matrix and collecting the eluted nucleic acids in a separate collecting vessel.

17. The method according to claim 16, wherein the volume of upper portion corresponds to a volume that is at least 5-fold the volume of lower portion.

18. The method according to claim 16, wherein the upper portion and lower portion independently of one another have a round or rectangular cross section.

19. The method according to claim 16, wherein the upper portion and lower portion independently of one another have a cylindrical or conical form.

20. The method according to claim 16, wherein the hollow body is manufactured of a plastic.

21. The method according to claim 16, wherein the plastic is selected from the groups consisting of polyolefins, bio-based plastics, polylactates, polyamides, polyimides, acetals, polyvinyl chloride, polytetrafluoroethylene, polyesters, polycarbonates, polymethyl(meth)acrylates, acrylonitrile butadiene styrene terpolymer (ABS), polystyrene, copolymers thereof, and mixtures thereof.

22. The method according to claim 16, wherein the hollow body is manufactured by an injection molding process, blow molding, casting technique with silicone forms, methods of rapid prototyping, fused deposition modeling, laser sintering or electron beam melting, or by means of forming machining methods.

23. The method according to claim 16, wherein the nucleic acid-binding matrix is a membrane, a fiber filter, frit and/or a particulate filter.

24. The method according to claim 16, wherein the nucleic acid-binding matrix is a silica membrane having a mean retention of 0.1 to 5μm measured according to DIN EN 1822-3.

25. The method according to claim 16, wherein the nucleic acid-binding matrix is immobilized on a carrier.

26. The method according to claim 16, wherein the predetermined breaking point is formed by a weakening line that substantially encircles the hollow body in a continuous form.

27. The method according to claim 16, wherein the predetermined breaking point is designed such that it enables a separation of the upper portion from the lower portion without the aid of tools.

28. The method according to claim 16, wherein the outlet opening is designed such that it is suitable for attachment to a vacuum chamber.

29. The method according to claim 16, wherein the nucleic acid yield is between 72 and 99% relative to the total amount of nucleic acids in the sample.

30. The method according to claim 29, wherein the total amount of nucleic acids in the sample is determined via a photometric measurement or quantitative real-time PCR.

31. The method according to claim 29, wherein the purification of nucleic acids is carried out in a clean-up procedure.

32. The method according to claim 16, wherein the nucleic acid yield is sufficient for subsequent PCR-Analysis.

33. A kit for purifying nucleic acids from a liquid nucleic acid-containing sample comprising a device for purifying nucleic acids composed of a one-piece hollow body comprising an upper portion having an inlet port and a lower portion having an outlet port, wherein at least one nucleic acid-binding matrix is arranged within the hollow body, wherein between the upper portion and the lower portion a predetermined breaking point is provided and the nucleic acid-binding matrix is arranged in the lower portion, and an operating manual for performing the method according to claim 29, and/or agents suitable for purifying nucleic acids.
